(19) United States
(12) Patent Application Publication
(10) Pub. No.: US 2025/0195413 A1
(43) Pub. Date: Jun. 19, 2025

(54) COMPOSITIONS AND METHODS FOR TREATING HAIR LOSS

(71) Applicant: Chemistry RX, FOLCROFT, PA (US)

(72) Inventor: Lars BRICHTA, Brooklyn, NY (US)

(21) Appl. No.: 19/072,723

(22) Filed: Mar. 6, 2025

Related U.S. Application Data
(63) Continuation-in-part of application No. 17/302,781, filed on May 12, 2021.
(60) Provisional application No. 63/023,275, filed on May 12, 2020.

(51) Int. Cl. A61K 9/00; A61K 31/506; A61K 31/519; A61K 31/58; A61K 47/10; A61K 47/44

(52) U.S. Cl. CPC A61K 9/0019; A61K 31/506; A61K 31/519; A61K 31/58; A61K 47/10; A61K 47/44

(57) ABSTRACT

Compositions and methods for treating hair loss in a patient are described herein. Injectable compositions including minoxidil, dutasteride, finasteride, tofacitinib citrate, or ruxolitinib phophate, a castor oil derivative, and a pharmaceutically acceptable carrier are described. Methods of treating hair loss in a patient in need thereof by administering the compositions by injection are also described.

### CROSS-REFERENCE TO RELATED APPLICATIONS

This application is a continuation-in-part of U.S. application Ser. No. 17/302,781, filed May 12, 2021, which claims the benefit of U.S. Provisional Application No. 63/023,275, filed May 12, 2020, the entirety of which is hereby incorporated by reference.

### SUMMARY OF THE INVENTION

Various embodiments of the invention are directed to compositions for treating hair loss in a subject. In embodiments, the composition includes one or more active agents, such as minoxidil, dutasteride, finasteride, tofacitinib, ruxolitinib, and salts thereof, for example tofacitinib citrate and ruxolitinib phosphate. In one embodiment, an injectable composition for treating hair loss in a subject including about 0.05% (w/w) to about 2% (w/w) of an active agent selected from the group consisting of minoxidil, dutasteride, finasteride, tofacitinib citrate, and ruxolitinib phosphate, about 5% (w/w) to about 50% (w/w) of a castor oil derivative, and a pharmaceutically acceptable carrier is disclosed herein. In other embodiments, the composition includes minoxidil and a secondary active agent in a pharmaceutically acceptable carrier. In some embodiments, the minoxidil may have a concentration of about 0.25% (w/w) to about 15% (w/w) based on the total composition. The secondary active agent of various embodiments may be finasteride, dutasteride, tofacitinib, ruxolitinib, tacrolimus, bimatoprost, latanoprost, spironolactone, aldactone, kenalog-10, kenalog-40, triamcinolone, azulfidine, sulfasalazine, sulfazine, and the like, including salts, and combinations thereof, and the secondary active agent may have a concentration of about 0.001% (w/w) to about 5% (w/w) of the total composition. In certain embodiments, the pharmaceutically acceptable carrier may be phosphate buffered saline solution, water, or saline, and in particular embodiments, the minoxidil, secondary active agent, and phosphate buffered saline solution, water, or saline may be the only components of the formulation.

In some embodiments, the composition may include a penetration enhancer such as, for example, carriers and vehicles, chemical penetration enhancers, diols, polyols, fatty acids, fatty alcohols, fatty acid esters, surfactants, pyrrolidones, and combinations thereof, and the penetration enhancer may have a concentration of about 1% (w/w) to about 20% (w/w) of the total composition. In some embodiments, the composition may include an antioxidant such as, for example, butylated hydroxytoluene, ascorbic acid, ascorbic palmitate, butylated hydroxyani sole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and the like and combinations thereof.

Further embodiments are directed to methods for treating hair loss by administering a therapeutically effective amount of a composition comprising minoxidil and a secondary active agent in a pharmaceutically acceptable carrier to a subject in need of treatment. In some embodiments, administering may include intradermal injection, and in some embodiments, administering may include injecting the composition into the dermis of the scalp.

In some embodiments, the minoxidil may have a concentration of about 0.25% (w/w) to about 15% (w/w) based on the total composition. The secondary active agent of various embodiments may be finasteride, dutasteride, tofacitinib, ruxolitinib, tacrolimus, bimatoprost, latanoprost, spironolactone, aldactone, kenalog-10, kenalog-40, triamcinolone, azulfidine, sulfasalazine, sulfazine, and the like and combinations thereof, and the secondary active agent may have a concentration of about 0.001% (w/w) to about 5% (w/w) of the total composition. In certain embodiments, the pharmaceutically acceptable carrier may be phosphate buffered saline solution, water, or saline, and in particular embodiments, the minoxidil, secondary active agent, and phosphate buffered saline solution, water, or saline may be the only components of the formulation.

In some embodiments, the composition may include a penetration enhancer such as, for example, carriers and vehicles, chemical penetration enhancers, diols, polyols, fatty acids, fatty alcohols, fatty acid esters, surfactants, pyrrolidones, and combinations thereof, and the penetration enhancer may have a concentration of about 1% (w/w) to about 20% (w/w) of the total composition. In some embodiments, the composition may include an antioxidant such as, for example, butylated hydroxytoluene, ascorbic acid, ascorbic palmitate, butylated hydroxyani sole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and the like and combinations thereof.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

All percentages, parts and ratios are based upon the total weight of the topical compositions and all measurements made are at about 25° C., unless otherwise specified.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g. “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are-within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “appreciable” is employed herein to refer to those agents of interest/compounds, salts, compositions, dosage forms, etc., which—within the scope of sound medical judgement—resulted in increased hair growth. An improvement in hair growth may be quantified by a SALT score or by a % regrowth measurement. A positive appreciable change in hair growth may not rise to a pharmaceutically acceptable or cosmetically acceptable determination.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

The term “SALT” refers to the Severity of Alopecia Tool, which is a statistical measurement that may be used by those skilled in the art to quantize change in the severity of alopecia in a patient or a sample of patients overall. A negative change in SALT score indicates an improvement in a subject's condition.

As used herein, the term “pharmaceutically acceptable carrier” refers to any of the standard pharmaceutical carriers including, but not limited to non-toxic solvent, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives.

The term “injectable” is a material having the properties necessary to administer the composition into a skin region of an individual using an injection device with any size needle suitable for administration.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed towards injectable compositions containing one or more active agents such as, for example, minoxidil, finasteride, dutasteride, tofacitinib, ruxolitinib, tacrolimus, bimatoprost, latanoprost, spironolactone, aldactone, kenalog-10, kenalog-40, triamcinolone, azulfidine, sulfasalazine, sulfazine, and the like, including salts, and combinations thereof. Such active agents can be provided in any amount capable of providing treatment. For example, the compositions of embodiments may include up to about 15% (w/w), about 0.05% (w/w) to about 2% (w/w), about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of active agent encompassed by these example ranges.

In certain embodiments, the composition may include minoxidil having a concentration of up to about 15% (w/w), about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of active agent encompassed by these example ranges, and a secondary active agent such as, for example, finasteride, dutasteride, tofacitinib, tacrolimus, ruxolitinib, bimatoprost, latanoprost, spironolactone, aldactone, kenalog-10, kenalog-40, triamcinolone, azulfidine, sulfasalazine, sulfazine, and the like, including salts, and combinations thereof having a concentration of up to about 5% (w/w), about 0.001% (w/w) to about 5% (w/w), about 0.005% (w/w) to about 5% (w/w), about 0.01% (w/w) to about 3% (w/w), about 0.05% (w/w) to about 2% (w/w), about 0.1% (w/w) to about 1% (w/w), about 0.001% (w/w) to about 1% (w/w), about 0.005% (w/w) to about 1% (w/w), or any range or individual concentration of active agent encompassed by these example ranges

Example compositions may include various known components. For example, in some embodiments, the composition may include a solvent such as water, isopropyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), Transcutol® solvent or diethylene glycol monoethyl ether, propylene glycol (PG), N-methyl-2 pyrrolidone (NMP), methylene chloride, diethyl ether, ethanol, acetonitrile, ethyl acetate, benzyl alcohol, a combination of natural oil; polyethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, ethosomes, azone, castor oil derivatives, such as ethoxylated castor oil, jojoba oil derivatives, corn oil derivatives, emu oil derivatives, and the like and combinations thereof. The solvent can be present in a concentration of up to about 70% (w/w), about 2% (w/w) to about 50% (w/w), about 5.0% (w/w) to about 15.0% (w/w)., about 6.0% (w/w) to about 10.0% (w/w), about 7.5% (w/w) to about 10.5% (w/w), about 8.0% (w/w) to about 10.0% (w/w), or any range or individual concentration of solvent encompassed by these example ranges.

In some embodiments, the compositions may include an antioxidant. Such antioxidant may be, for example, butylated hydroxytoluene, ascorbic acid, ascorbic palmitate, butylated hydroxyani sole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include a UV-absorbing compound such as, for example, glyceryl PABA, padimate O, roxadimate, dioxybenzone, oxybenzone, sulisonbenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, bisoctrizole, and the like and combinations thereof. The amount of UV-absorbing compound may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

The compositions of the various embodiments described herein may include one or more penetration enhancers. The penetration enhancer in the compositions of various embodiments described above may be present in an amount about 0.5% (w/w) to about 40% (w/w), about 1% (w/w) to about 20% (w/w), about 5% (w/w) to about 15% (w/w) based on the total composition or any range or individual concentration encompassed by these example ranges.

Other embodiments of the invention include methods for treating hair growth disorders or hair loss by administering the compositions described above. Such methods are not limited to particular indications; however, the compositions described herein can be particularly useful for treating alopecia areata, alopecia totalis, alopecia universalis, vitiligo, and graft versus host disease. Other indications that can be treated by administering the compositions of various embodiments, include telogen effluvium, tinca capitis (dermatophytosis), hypotrichosis, hereditary hypotrichosis simplex, frontal fibrosing alopecia, cicatricial alopecia, lichen planopilaris, ring alopecia, chemotherapy induced alopecia, and the like.

The methods of various embodiments may include the steps of administering a composition of the various embodiments described above to an area of skin on a subject in need of treatment. In some embodiments, the step of administering can be carried out one or two times per week, one, two, or three times per month, one to four times per year, and the like and any time period encompassed by these examples. In certain embodiments, administering can be carried out the prescribed number of times per day for one week to six months.

In certain embodiments, administering can be carried out by intradermal injection. Intradermal delivery generally requires a smaller volume dose of the compositions than other delivery techniques such as topical delivery. An intradermal injection is made by delivering the substance into the epidermis and upper layer of the dermis. In certain embodiments, the methods may include administering the composition below the stratum corneum of the patient, for example, to the dermis or hypodermis. For example, a device may inject the composition past the stratum corneum of the skin and into the epidermis or into the dermis or into the subcutaneous tissue (hypodermis) where the composition can act on the tissues and cells that grow hair.

Intradermal delivery may reduce absorption of the formulation or active agents in the formulation by subcutaneous layers and into blood vessels where the active agents are diluted and carried away from the site of treatment. Injection may also reduce side effects. Although there is considerable variation in the skin thickness, both between individuals and within the same individual at different sites of the body, the epidermis has a thickness between 500-200 microns and the dermis, the inner and thicker layer of the skin, has a thickness between 1.5-3.5 mm.

The step of administering can be carried out by various means. For example, administering can be accomplished by injecting the composition to the subdermal and subcutaneous areas of the scalp in need of treatment. In some embodiments, administering may include applying mechanical force or energy to the skin of the subject to facilitate delivery. For example, administering includes injecting the composition into the skin of the subject using microneedles. In further embodiments, administering can be carried out using a tattoo machine or other machines for subcutaneously injecting substances into a subject. As is known in the art, certain means for administering may require the use of particular components of the formulation. Such components are described above and can be appropriately incorporated into the compositions.

The term “microneedle injection” refers to methods for injection of the compositions described above to the scalp using, for example, microneedles such as 19- or 21-gauge needles, micro-coring needles, and the like and combinations thereof.

In some embodiments, a microneedle array can be used to administer the compositions of the invention. The microneedle array may be a roller or a plate and can deliver the composition to a skin area of about 1.5 cm×1.5 cm to about 15 cm×15 cm to a depth of about 100 microns to about 4000 microns. In some embodiments, the microneedle array may include a luer-lock fitting that can accommodate a syringe that acts as a reservoir for the composition and allows the composition to be delivered to each of the microneedles in the array at a relatively similar rate. Volumes of the compositions formulated for injection and included in the syringe can be, for example, about 0.1 ml to about 10 ml, about 0.1 ml to about 5 ml, about 0.1 ml to about 2 ml, about 0.1 ml to about 1 ml, about 0.4 ml, about 0.5 ml, about 0.6 ml, about 0.7 ml, about 0.8 ml, about 0.9 ml, about 1.0 ml or more.

Various devices and for microneedle injection can be used in embodiments of the invention, including, for example, the devices and methods described in U.S. Publication Nos. US 2011/0130711, US 2011/0130748, or US 2011/0130706, each of which is incorporated by reference herein in its entirety, may be used in accordance with the invention.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

The following formulations were prepared for delivery by microneedle injection to the scalp of patients having alopecia areata.
Example Formulation 1:0.5% minoxidil in water for intradermal injection.
Example Formulation 2:0.5% minoxidil/0.005% finasteride in water for intradermal injection.
Example Formulation 3:0.5% minoxidil/0.001% dutasteride in water for intradermal injection.

### Example 2

Treatment options for hair loss are limited with oral and topical treatments having side effects and limitations, including messy to apply and patient compliance. Oral treatments like dutasteride capsules and finasteride tablets are often associated with highly undesirable side effects, such as decreased libido and erectile dysfunction in men. Oral minoxidil therapy with minoxidil tablets can be associated with unwanted hair growth on body parts other than the scalp (face, body, arms, legs), or result in lower leg edema, in women as well as men. These side effects are bothersome to patients and substantially limit the use of oral treatments for hair loss. Topical treatments are associated with the major disadvantage of having to penetrate through the scalp first, in order to reach the hair follicles as their target structure. Hair follicles are not located on the scalp surface. Instead, they are located about 3 mm deep in the skin. It is typically difficult to ensure that a particular drug penetrates to the hair follicle after topical application, and because of that, penetration enhancers are mandatory for topical formulations. However, it is unknown what fraction of a drug prepared as a topical formulation actually makes it to the hair follicle, resulting in a large variation of clinical outcomes. Moreover, given that only a small fraction of drug will get to the hair follicle after topical administration, topicals are typically considered only for mild cases of hair loss. Topicals can also be associated with substantial side effects that arise from the inactive components needed in such formulations. Irritation can occur, particularly from propylene glycol, but also from ethanol and/or penetration enhancers such as DMSO, limiting the use of topicals. Similarly to oral minoxidil, topical minoxidil can also cause unwanted hair growth particularly on the face of female patients because some of the liquid topical formulation may run down from the scalp, onto the face. In addition, topical preparations can be problematic cosmetically, as particularly female and male patients with longer hair dislike the daily or twice-daily use of topicals because they make long hair look and feel “greasy” and “thin”, which is unwanted, and most patients with longer hair are unable to wash their hair daily or twice daily as it is very inconvenient and time-consuming. Therefore, many patients may try topical formulations but then stop using them again. The injectable compositions and methods disclosed herein solve these problems.

Other example injectable compositions for treating hair loss include about 0.05% (w/w) to about 2% (w/w) of an active agent such as minoxidil, dutasteride, finasteride, tofacitinib citrate, or ruxolitinib phosphate, about 5% (w/w) to about 50% (w/w) of a castor oil derivative, and a pharmaceutically acceptable carrier, such as water, and optionally a co-solvent. Optionally, the composition may also include benzyl alcohol, polyethylene glycol (PEG), and/or a preservative.

Example compositions are:
1) about 0.75% (w/w) minoxidil, about 0.9% (w/w) benzyl alcohol, about 25% (w/w) polyethylene glycol (PEG) 400, about 15% (w/w) castor oil derivative, such as polyoxyl 40 hydrogenated castor oil, and about 58.35% (w/w) water;
2) about 0.1% (w/w) dutasteride, about 0.9% (w/w) benzyl alcohol, about 2% (w/w) PEG 400, about 30% (w/w) castor oil derivative, such as polyoxyl 40 hydrogenated castor oil, and about 67% (w/w) water;
3) about 0.1% (w/w) finasteride, about 0.9% (w/w) benzyl alcohol, about 2% (w/w) PEG 400, about 30% (w/w) castor oil derivative, such as polyoxyl 40 hydrogenated castor oil, and about 67% (w/w) water;
4) about 0.3% (w/w) tofacitinib citrate, about 0.9% (w/w) benzyl alcohol, about 23.5% (w/w) PEG 400, about 20% (w/w) castor oil derivative, such as polyoxyl 40 hydrogenated castor oil, and about 55.3% (w/w) water, and
5) about 0.3% (w/w) ruxolitinib phosphate, about 0.9% (w/w) benzyl alcohol, about 25% (w/w) PEG 400, about 14% (w/w) castor oil derivative, such as polyoxyl 40 hydrogenated castor oil, and about 59.8% (w/w) water.

What is claimed is:

1. An injectable composition for treating hair loss in a subject comprising about 0.05% (w/w) to about 2% (w/w) of an active agent selected from the group consisting of minoxidil, dutasteride, finasteride, tofacitinib citrate, and ruxolitinib phosphate, about 5% (w/w) to about 50% (w/w) of a castor oil derivative, and a pharmaceutically acceptable carrier.

2. The composition of claim 1, wherein the composition further comprises polyethylene glycol.

3. The composition of claim 2, wherein the composition further comprises about 1% (w/w) to about 30% (w/w) polyethylene glycol.

4. The composition of claim 1, wherein the composition further comprises benzyl alcohol.

5. The composition of claim 4, wherein the composition comprises about 0.5% (w/w) to about 1% (w/w)) benzyl alcohol.

6. The composition of claim 1, wherein the composition further comprises a preservative.

7. The composition of claim 1, wherein the pharmaceutically acceptable carrier is phosphate buffered saline solution, water, or saline.

8. The composition of claim 7, wherein the pharmaceutically acceptable carrier is water.

9. The composition of claim 1, wherein the composition comprises about 0.5% (w/w) to about 1% (w/w) minoxidil.

10. The composition of claim 1, wherein the composition comprises about 0.05% (w/w) to about 0.5% (w/w) dutasteride.

11. The composition of claim 1, wherein the composition comprises about 0.1% (w/w) to about 1% (w/w) tofacitinib citrate.

12. The composition of claim 1, wherein the composition comprises about 0.05% (w/w) to about 1% (w/w) ruxolitinib phosphate.

13. The composition of claim 1, wherein the composition comprises about 0.05% (w/w) to about 0.5% (w/w) finasteride.

14. A method for treating hair loss comprising administering a therapeutically effective amount of a composition comprising about 0.05% (w/w) to about 2% (w/w) of an active agent selected from the group consisting of minoxidil, dutasteride, tofacitinib, and ruxolitinib, about 5% (w/w) to about 50% (w/w) of a castor oil derivative, and a pharmaceutically acceptable carrier to a subject in need of treatment.

15. The method of claim 14, wherein administering comprises intradermal injection.

16. The method of claim 14, wherein administering comprises injecting the composition into the dermis of the scalp.

17. The method of claim 14, wherein the composition further comprises polyethylene glycol.

18. The method of claim 14, wherein the composition further comprises benzyl alcohol.

19. The method of claim 14, wherein the pharmaceutically acceptable carrier is phosphate buffered saline solution, water, or saline.

20. The method of claim 14, wherein the composition further comprises a preservative.

21. The method of claim 14, wherein the subject has a disorder selected from the group consisting of alopecia areata, alopecia totalis, alopecia universalis, vitiligo, graft versus host disease, telogen effluvium, tinea capitis (dermatophytosis), hypotrichosis, hereditary hypotrichosis simplex, frontal fibrosing alopecia, cicatricial alopecia, lichen planopilaris, ring alopecia, and chemotherapy induced alopecia.
